(19) United States
(12) Patent Application Publication
(10) Pub. No.: US 2018/0284114 A1
(43) Pub. Date: Oct. 4, 2018

(54) METHODS FOR PROCESSING BIOPOLYMERIC ARRAYS

(71) Applicant: ARIZONA BOARD OF REGENTS ON BEHALF OF ARIZONA STATE UNIVERSITY, Scottsdale, AZ (US)

(72) Inventor: Stephen Johnston, Tempe, AZ (US)

(21) Appl. No.: 15/764,143

(22) PCT Filed: Sep. 30, 2016
(86) PCT No.: PCT/US16/54756

§ 371 Date: Mar. 28, 2018

Related U.S. Application Data
(60) Provisional application No. 62/236,390, filed on Oct. 2, 2015.

(51) Int. Cl. G01N 33/543

(52) U.S. Cl. CPC G01N 33/54393

(57) ABSTRACT

Provided herein are methods for processing biopolymeric arrays, where conditions suitable for forming complexes of immobilized biopolymers (e.g., peptides) and target molecules of a sample can be varied independently of conditions suitable for detecting such complexes using a secondary detection reagent.

### CROSS-REFERENCE TO RELATED APPLICATIONS

This application claims the benefit of U.S. Provisional Patent Application No. 62/236,390, filed Oct. 2, 2015, which is incorporated herein by reference as if set forth in its entirety.

### STATEMENT REGARDING FEDERALLY SPONSORED RESEARCH

Not applicable.

### BACKGROUND OF THE INVENTION

### 1. Field of the Invention

This application relates to methods of assaying for targets in an unknown sample. More particularly, this document provides methods of processing arrays comprising a plurality of support-bound biopolymers such as peptides and polypeptides.

### 2. Background

Arrays of biopolymeric capture agents are useful for determining the relative amounts of several analytes, e.g., proteins, in whole blood, serum, and other biological samples. For example, peptide arrays are useful for medical diagnostics and various biomedical applications such as screening patient samples for the presence or absence of tumor-specific antigens or disease-specific antibodies.

The applicability of microarray technology for large scale proteomics studies remains limited due to the difficulty and costs associated with producing, processing, and screening peptide arrays. Standard protocols for processing peptide arrays involve first applying a polypeptide-containing sample to a substrate comprising bound peptides or polypeptides (e.g., antibodies), followed by incubations with detection molecules such as labeled antibodies and washes to reduce non-specific binding. With every wash or incubation after contacting the biological sample to the array, there is risk of losing detection molecules or peptides from the array or losing the interaction between array-bound peptides and target molecules. Moreover, standard peptide array processing protocols do not permit varying secondary binding conditions independently of primary sample binding conditions. Accordingly, there remains a need in the art for improved methods for processing and screening peptide arrays.

### SUMMARY OF THE INVENTION

In a first aspect, provided herein is a method for detecting a target molecule in a sample. The method comprises, or consists essentially of, (a) contacting a sample that comprises, or is at least suspected of comprising, a target molecule of interest to an array comprising a plurality of biopolymers immobilized on a support, wherein contacting occurs under conditions conducive to the target molecule binding to one or more immobilized biopolymers of the plurality; (b) removing excess or unbound sample from the sample-contacted array; (c) contacting a fixative agent to the sample-contacted array, whereby the fixative agent fixes any biopolymer-target molecule binding complexes; (d) removing the fixative agent from the fixative-contacted array; (e) contacting the array of step (d) to a secondary detection reagent under conditions that promote binding of the secondary detection reagent to biopolymer-target molecule binding complexes; and (f) measuring the secondary detection reagent-contacted array to detect signals from labeled target molecules bound to the one or more biopolymers.

The conditions of step (a) and the conditions of step (e) can be varied independently. The fixative agent can be an organic solvent. The organic solvent can be isopropyl alcohol. In some cases, the target molecule is selected from the group consisting of an antibody, peptide, polypeptide, and antigen. The secondary detection reagent can be selected from the group consisting of an antibody, peptide, polypeptide, antibody fragment, enzyme substrate, and dye. The biological sample can be selected from the group consisting of whole blood, serum, plasma, urine, saliva, peritoneal fluid, and tissue. The plurality of biopolymers can comprise peptides. The plurality of biopolymers can comprise a random peptide array. The random peptide array can be synthesized in situ on the support. In some cases, the method further comprises the step of identifying the biopolymer of the plurality that binds the target molecule.

These and other features, aspects, and advantages described herein will become better understood upon consideration of the following drawings, detailed description, and appended claims.

### DETAILED DESCRIPTION OF THE INVENTION

All publications, patents, and patent applications mentioned in this specification are herein incorporated by reference to the same extent as if each individual publication, patent, or patent application was specifically and individually indicated to be incorporated by reference.

Disclosed herein is the discovery of methods for processing biopolymeric arrays in which binding and washing conditions for various detection reagent (e.g., primary detection reagent, secondary detection reagent) can be varied independently. Since epitope affinity and binding kinetics vary greatly between antibodies, each binding reaction is essentially an independent experiment and may require completely different buffers, antibody reagents, and incubation conditions. Without being bound by any theory or mechanism, it is believed that contacting an array with a fixative agent following contact of the array with the sample prevents disruption of any ligand/analyte (e.g., peptide-target molecule) binding complexes on the array and any signal loss resulting from the disruption. Thus, the methods provided herein are improved over existing protocols in that conditions suitable for binding and detecting a primary sample or detection reagent (e.g., primary antibody) can be varied independently of conditions suitable for binding and detecting a secondary detection reagent (e.g., secondary antibody).

Methods

Accordingly, provided herein are methods for processing biopolymeric arrays and for using such methods to identify antibodies and proteins of analytical or diagnostic significance. As used herein, the term “processing” encompasses any array manipulation protocol including use of a biopolymeric array in an array-based method of analyzing a sample. As described herein, the methods are advantageous over standard processing methods in that the incubation and wash steps for each detection reagent or capture agent can be independently optimized to maximize specific protein-protein interactions without degrading the quality of interactions between biopolymers and the sample, or between biopolymer-target molecule complexes and a secondary detection reagent (e.g., labeled secondary antibody).

As used herein, the term “biopolymeric array” refers to a plurality of biological molecules (e.g., peptides, polypeptides) arrayed on and attached to a support. The term “attached,” as in, for example, a substrate surface having a plurality of peptides “attached” thereto, includes covalent binding, adsorption, and physical immobilization. The term “peptide” as used herein refers to a plurality of amino acids joined together in a linear or circular chain. Peptide arrays are also commonly known as peptide chips, peptide microarrays, or peptide epitope microarrays. In some cases, a peptide array comprises an antibody or an antibody fragment comprising at least one polypeptide chain. In some cases, the polypeptide chain is not a full-length antibody chain and, rather, is (i) a Fab fragment, which is a monovalent fragment consisting of the variable light (V_{L}), variable heavy (V_{H}), constant light (C_{L}) and constant heavy 1 (C_{H}1) domains; (ii) a F(ab′)2 fragment, which is a bivalent fragment comprising two Fab fragments linked by a disulphide bridge at the hinge region; (iii) a heavy chain portion of an Fab (Fd) fragment, which consists of the V_{H} and C_{H1} domains; (iv) a variable fragment (Fv) fragment, which consists of the V_{L} and V_{H} domains of a single arm of an antibody, (v) a domain antibody (dAb) fragment, which comprises a single variable domain; (vi) an isolated complementarity determining region (CDR); (vii) a Single Chain Fv Fragment; (viii) a diabody, which is a bivalent, bispecitc antibody in which V_{H} and V_{L} domains are expressed on a single polypeptide chain, an engineered constant domain such as Ckappa or Clambda, C_{H}1, C_{H}2, C_{H}3 or C_{H}4.

At each immobilized spot on an array is a homogeneous or heterogeneous set of “capture” or query molecules such as antibodies, a cell or phage lysate, a recombinant protein or peptide, a drug, or nucleic acid. Generally, peptide arrays are queried with a probe (e.g., labeled antibody or ligand) or an unknown biologic sample (e.g., cell lysate, blood, serum sample) containing analytes of interest. By tagging the query molecules with a signal-generating moiety, a pattern of positive and negative spots is generated. For each spot, the intensity of the signal is proportional to the quantity of applied query molecules bound to the bait molecules. In general, the identity of which query molecules occupy which area of an array is usually either known as a result of the synthesis process or determinable as a result of an encoding process. See for example, U.S. Patent Publication No. 2013/0079242, which is incorporated herein as if set forth in its entirety.

In preferred embodiments, methods for processing a biopolymeric array comprise the steps of contacting a sample that comprises, or is at least suspected of comprising, one or more target molecule (e.g., antibody) of interest to a biopolymeric array, where the biopolymeric array comprises a plurality of biopolymers immobilized on a substrate surface; washing excess or unbound sample from the array; contacting a fixative agent to the sample-contacted array, whereby the fixative agent fixes any biopolymer-target molecule binding complexes; removing the fixative agent from the fixative contacted-array; contacting the fixative-contacted array to a secondary detection reagent under conditions conducive for binding of the secondary detection reagent to peptide-target molecule binding complexes; and detecting binding of the secondary detection reagent to peptide-target molecule binding complexes.

In the first step, a sample that is at least suspected to have (if not known to include) a target molecule of interest is contacted to an array comprising a plurality of binding biopolymer agents that includes a binding agent (ligand) specific for the target molecule of interest under conditions conducive for the target molecule to bind to its respective binding pair member that is present on the array. Thus, if the target molecule of interest is present in the sample, it binds to the array at the site of its complementary binding member and a complex is formed on the array surface.

In exemplary embodiments, the sample is contacted to the array in a manner sufficient to bring the sample into contact with the surface immobilized biopolymeric agents of the array. As such, the array may be placed on top of the sample, the sample may be deposited on the array surface, the array may be immersed in the sample, etc.

Following contact of the array and the sample, the resultant sample contacted or exposed array is then maintained under conditions conducive for and for a sufficient period of time for any binding complexes between members of specific binding pairs to occur. The reagents used in each of steps of the methods described herein and suitable conditions for their use will vary depending on the particular application. In many embodiments, the duration of this step is at least about 10 min long, often at least about 20 min long, and may be as long as 30 minutes or longer, but generally does not exceed about 72 hours.

In exemplary embodiments, a primary sample comprising target polypeptide or peptide is contacted to the peptide array under conditions suitable for binding of a peptide of the array and one or more target molecules of the primary sample. Binding of an array-bound peptide to a target molecule, if it occurs, forms a peptide-target molecule complex. Stringency of binding can be adjusted by varying the salts, ionic strength, organic solvent content, and/or temperature at which peptide array members are contacted with the target polypeptide.

After contacting the primary sample to the peptide array and formation, if any, of a peptide-target molecule complex, excess or unbound primary sample is washed away. Preferably, excess or unbound primary sample is washed away under conditions suitable to retain any peptide-target molecule complexes. Generally, incubation and washing parameters that can be varied include, without limitation, salts, ionic strength, organic solvent content, temperature, length of wash or incubation time.

The methods provided herein further comprise contacting a fixative agent to the array and, thus, to array-bound peptide-target molecule complexes. As used herein, the term “fixative agent” refers to reagents that fix or stabilize binding complexes present in the features and prevent disassociation of the complex components. Useful fixative agents include, without limitation, isopropyl alcohol (also known as isopropanol and 2-propanol), methanol, and other water-miscible organic solvents (e.g., acetone, tert-butanol, n-propanol, ethanol, dioxane). In exemplary embodiments, isopropyl alcohol applied to peptide-target molecule binding complexes on the array, thereby fixing (stabilizing) the complexes before subjecting the array to further washes or incubations.

Next, the peptide array is washed to remove the fixative agent and contacted with a buffer suitable for binding and detection of a secondary detection regent (e.g., secondary antibody). Any appropriate incubation and washing parameters can be used to remove the fixative agent and to incubate the array with a secondary detection reagent. Parameters that that can be varied include, without limitation, salts, ionic strength, organic solvent content, temperature, length of wash or incubation time.

Following the above-described fixation step, the presence of any binding complexes on the array surface is then detected. In some cases, detecting comprises use of a signal production system such as, for example, an isotopic or fluorescent label present on the analyte. Binding of target molecules in a sample to one or more peptides of the plurality on the array can be detected using a secondary detection reagent specific for a target molecule or, if applicable, a primary detection reagent (e.g., primary antibody). Accordingly, some methods provided herein further comprise contacting a secondary antibody having the needed specificity (e.g., anti-IgG (including any of the subtypes, such as IgG1, IgG2, IgG3, and IgG4), anti-IgA, anti-IgM) to the array and maintained under conditions suitable for its binding with minimal non-specific interactions. The secondary detection reagent (e.g., secondary antibody) is incubated for an amount of time sufficient for the formation of a complex of target molecule, peptide, and detection reagent. The secondary antibody is usually labeled and can bind to all peptides in the sample being analyzed of a particular isotype. Different secondary antibodies can be used having different isotype specificities. In some cases, the secondary antibody is tagged by a fluorescence label that can be detected by a fluorescence scanner. Other detection methods include chemiluminescence or autoradiography.

After any excess detection reagent is removed, the presence of target molecules in the sample is then deduced or determined from the detection of binding complexes on the support. Preferably, the presence of a target molecule is determined by observation of a signal produced by a detectable label, indicating the formation of a complex of target molecule, peptide, and detection reagent. The results may either be qualitative, by simple observation of the visible signal, or may be quantified by comparing with a control sample. For example, the signal intensity can be measured and compared to that of a reference sample having known amounts of a molecule.

As used herein, the term “sample” refers to biological samples that have been provided by a human patient or an animal (e.g., blood, lymph, urine, saliva, sputum, other bodily secretions, cells, and tissue specimens) as well as non-biological samples (e.g., samples prepared in vitro comprising varying concentrations of a target molecule of interest in solution).

Biopolymeric arrays can be designed and produced to analyze and characterize a variety of biological samples, including clinical, veterinary, forensic, laboratory, and other samples. As with conventional diagnostics, the arrays can be used to identify particular analytes within samples, for example, analytes associated with particular disease. However, the methods can also be used to provide a binding profile of different compounds characterizing a sample. The binding profile can represent the aggregate interactions of the compounds with different components in the sample, and can be characteristic of a particular disease, stage of disease or lack of disease.

The terms “non-specific binding” and “background binding” are well known terms in the biological arts and generally refer to unintended, passive binding of, for example, an antibody to a substrate used in the assay or to a contaminant present in the assay. In contrast, “specific binding” refers to the desired interaction of an antibody to the appropriate antigen.

Any appropriate biopolymeric array can be used for the methods provided herein. In some cases, the arrayed biopolymers are peptides. Such arrays can be prepared via stepwise in situ synthesis on the support. In some cases, the plurality of peptides comprises completely random peptide sequences, peptides designed de novo, or peptides derived from a protein, or a fragment or domain of a protein. In exemplary embodiments, the peptide array comprises a random peptide library immobilized on a support (e.g., a solid support). In other cases, peptide libraries can be generated by phage display methods. See, e.g., Devlin, WO 91/18980.

The term “support” as used herein refers to any material having a surface onto which one or more fluids may be deposited. The support may be constructed in any of a number of forms such as wafers, slides, well plates, membranes, for example. In addition, the support may be porous or nonporous as may be required for deposition of a particular fluid. Suitable support materials include, but are not limited to, those supports that are typically used for solid phase chemical synthesis, e.g., polymeric materials (e.g., polystyrene, polyvinyl acetate, polyvinyl chloride, polyvinyl pyrrolidone, polyacrylonitrile, polyacrylamide, polymethyl methacrylate, polytetrafluoroethylene, polyethylene, polypropylene, polyvinylidene fluoride, polycarbonate, divinylbenzene styrene-based polymers), agarose (e.g., Sepharose®), dextran (e.g., Sephadex®), cellulosic polymers and other polysaccharides, silica and silica-based materials, glass, and functionalized glasses, ceramics, and such substrates treated with surface coatings, e.g., with microporous polymers (particularly cellulosic polymers such as nitrocellulose), microporous metallic compounds (particularly microporous aluminum), antibody-binding proteins (available from Pierce Chemical Co., Rockford Ill.), bisphenol A polycarbonate, or the like. In some cases, array supports are porous surfaces or gels such as methacrylates, acrylamides, sugar polymers, cellulose, silicates, and other fibrous or stranded polymers.

The methods of this embodiment of the present invention find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label present on the analyte, etc. The presence of the analyte in the sample is then deduced from the detection of binding complexes on the substrate surface.

For example, the methods provided herein find use in immunosignaturing (i.e., the process of detecting immunosignatures). Immunosignaturing comprises contacting a sample (e.g., blood) a large number of peptides or other molecular heteropolymers each associated with a feature on a surface. Antibodies in the sample bind differentially to the query molecules at each feature, thus forming a pattern of binding that provides a detailed insight into the molecular recognition profile of the antibodies in the blood. See, e.g., Stafford and Johnson, Exp. Rev. Mol. Diagn. 11:5-8 (2011). The concept is that any change in health is likely to be represented by a change in this molecular recognition profile. Such profiles can be used in various analytical methods to further characterize the sample. See for example, U.S. Patent Publication No. 2013/0079242, which is incorporated herein as if set forth in its entirety.

In some cases, the methods provided herein are useful for performing immunoassays. As used herein, the terms “immunoassay” and “antibody-based assay” may be used interchangeably and refer to any technique based on the interaction between an antibody and its corresponding antigen. Such techniques are based on the unique ability of an antibody to bind with high specificity to one or a very limited group of similar molecules. The term “antigen” refers to a molecule that binds to an antibody. Immunoassays can be carried out using either the antigen or antibody as the “capture” molecule to “entrap” the other member of the antibody-antigen pairing. As used herein, the terms “immunoassay” and “antibody-based assay” also refer to those assays that utilize antibodies for the detection of a non-protein biomarker in a biological sample (e.g., nucleic acids or metabolites of biochemical reactions).

Exemplary immunoassays include, without limitation, a radioimmunoassay (MA), an enzyme immunoassay (EIA), an enzyme-linked immunosorbent assay (ELISA), a fluorescent immunoassay, and a chemiluminescent immunoassay. One of skill in the art is capable of selecting and implementing the appropriate immunoassay under a particular set of circumstances, as well performing these immunoassays and interpreting their results. Immunoassays may produce qualitative or quantitative results depending on the particular method of detection selected.

Examples of detectable substances include various enzymes, prosthetic groups, fluorescent materials, luminescent materials, bioluminescent materials, radioactive materials, and nanoparticles. Exemplary suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin; a detectable luminescent material that may be couple to an antibody includes but is not limited to luminol; examples of bioluminescent materials include luciferase, luciferin, and aequorin. Methods for conjugating or coupling a detectable substance to an antibody and for detecting these agents are well known in the art.

Articles of Manufacture

Kits for use in array processing protocols, such as analyte detection assays, as described above, are also provided. The kits at least include a fixation agent as described herein. Such kits may comprise one or more containers containing a fixation agent and various other reagents (e.g., reducing reagents, denaturing reagents, dephosphorylating reagents, alkylating reagents and/or reagents for chemically or enzymatically cleaving a peptide or protein). Such kits typically also include instructions for use in practicing array-based assays as provided herein in which a fixation step is performed.

The kits may further comprise one or more peptide arrays for performing the analysis. Alternatively, the substrate and/or some of the reagents (e.g., reducing reagents, denaturing, deglycosylating reagents, dephosphorylating reagents, alkylating reagents and/or reagents for chemically or enzymatically cleaving the peptide or protein) may be provided separately from the kit.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g., via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{th} Ed., W. H. Freeman Pub., New York, N.Y.; and Berg et al. (2002) Biochemistry, 5^{th} Ed., W.H. Freeman Pub., New York, N.Y., all of which are herein incorporated in their entirety by reference for all purposes.

“Determining,” “measuring,” “assessing,” “assaying” and like terms are used interchangeably and can include both quantitative and qualitative determinations. Assessing may be relative or absolute. “Assessing the presence of” includes determining the amount of something present, as well as determining whether it is present or absent.

Note that as used herein and in the appended claims, the singular forms “a,” “an,” and “the” include plural referents unless the context clearly dictates otherwise. Thus, for example, reference to “an array” refers to one or more such arrays, and reference to “the method” includes reference to equivalent steps and methods known to those skilled in the art, and so forth.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

### Examples

Based on the methods described herein, changes in immunosignature performance were measured using 6 sets of “before” QC data and 7 sets of “after” QC data, i.e., before and after isopropyl alcohol washes. The “before” data consisted of 12 case and 12 control samples from persons diagnosed with breast cancer within one year (case) or not diagnosed with breast cancer for the duration of the Prostate Lung Colorectal and Ovarian Cancer Clinical Trial funded by the National Institute of Health (NIH). Samples were processed on the 125K arrays. Each slide had 12 case and 12 control samples randomly placed across the slide. Immunosignatures were gathered and analyzed.

In summary, the dynamic range “before” was 2.41 logs, the evenness score “before” was 1.8141 (1 is best), and the Leave Out One-Cross Validation (LOO-CV) “before” was 100%. In comparison, the dynamic range “after” was 3.02 logs, the evenness score “after” was 1.2166 (1 is best), and the LOO-CV “after” was 100%. Thus, there is no loss of signal with isopropyl alcohol and more dynamic range.

By way of further example, reduction in plaque forming units (pfu) with vaccinia virus was done using 30 hour cultures of freshly inoculated vaccinia. 10̂8 and 10̂9 virus particles were spiked into 2 ml of incubation buffer+human serum, covering an entire slide (24 arrays). One slide was not fixed using isopropyl alcohol (control slide) and one slide was washed for 20 seconds with 95% isopropyl alcohol (IPA slide). Measurements of virus titer were done using the 10̂9 spiked sample. Recovery measurements were done by testing for plaque forming units from the incubation buffer, wash buffer 1 and wash buffer 2. The incubation buffer should not harm pox virus. Washes can only accumulate pox virus that were retained temporarily on the arrays, however IPA was also tested on pox viability and a near-100% reduction in pfu was noted when directly added to virions in serum.

Incubation buffer recovery: 86% recovery control slide, 87% recovery IPA slide; wash buffer 1: 0.0831% recovery control slide, 0.0000% recovery IPA slide; and wash buffer 2: 0.0001 recovery control slide, 0.0000% recovery IPA slide.

Although the embodiments and examples are described in considerable detail with reference to certain methods and materials, one skilled in the art will appreciate that the disclosure herein can be practiced by other than the described embodiments, which have been presented for purposes of illustration and not of limitation. Therefore, the scope of the appended claims should not be limited to the description of the embodiments contained herein.

We claim:

1. A method for detecting a target molecule in a sample, the method comprising
(a) contacting a sample that comprises, or is at least suspected of comprising, a target molecule of interest to an array comprising a plurality of biopolymers immobilized on a support, wherein contacting occurs under conditions conducive to the target molecule binding to one or more immobilized biopolymers of the plurality;
(b) removing excess or unbound sample from the sample-contacted array;
(c) contacting a fixative agent to the sample-contacted array, whereby the fixative agent fixes any biopolymer-target molecule binding complexes;
(d) removing the fixative agent from the fixative-contacted array;
(e) contacting the array of step (d) to a secondary detection reagent under conditions that promote binding of the secondary detection reagent to biopolymer-target molecule binding complexes; and
(f) measuring the secondary detection reagent-contacted array to detect signals from labeled target molecules bound to the one or more biopolymers.

2. The method of claim 1, wherein the conditions of step (a) and the conditions of step (e) can be varied independently.

3. The method of claim 1, wherein the fixative agent is an organic solvent.

4. The method of claim 4, wherein the organic solvent is isopropyl alcohol.

5. The method of claim 1, wherein the target molecule is selected from the group consisting of an antibody, peptide, polypeptide, and antigen.

6. The method of claim 1, wherein the secondary detection reagent is selected from the group consisting of an antibody, peptide, polypeptide, antibody fragment, enzyme substrate, and dye.

7. The method of claim 1, wherein the biological sample is selected from the group consisting of whole blood, serum, plasma, urine, saliva, peritoneal fluid, and tissue.

8. The method of claim 1, wherein the plurality of biopolymers comprises peptides.

9. The method of claim 1, wherein the plurality of biopolymers comprises a random peptide array.

10. The method of claim 9, wherein the random peptide array is synthesized in situ on the support.

11. The method of claim 1, further comprising the step of identifying the biopolymer of the plurality that binds the target molecule.
